(19) United States
(12) Patent Application Publication
(10) Pub. No.: US 2006/0130248 A1
(43) Pub. Date: Jun. 22, 2006

(54) Easily removable water resistant cosmetic makeup compositions

(75) Inventors: Karl Pays, Saint-Maurice (FR); Olivier Raineau, Paris (FR); Yohann Bichon, Maisons-Alfort (FR)

Correspondence Address: OLIFF & BERRIDGE, PLC, P.O. BOX 19928, ALEXANDRIA, VA, 22320, US

(73) Assignee: L'OREAL, PARIS (FR)

(21) Appl. No.: 11/312,583

(22) Filed: Dec. 21, 2005

Related U.S. Application Data
(60) Provisional application No. 60/642,992, filed on Jan. 12, 2005.

(30) Foreign Application Priority Data
Dec. 21, 2004 (FR) 04 53120

(51) Int. Cl. A61K 8/81

(52) U.S. Cl. 8/406; 424/70.16

(57) ABSTRACT

A cosmetic composition for making up keratin materials includes at least one polyelectrolyte, at least one surfactant having an HLB of at least about 6 at 25° C., and at least one dyestuff. A leave-on cosmetic composition for making up and/or nontherapeutic care of keratin fibers includes at least one polyelectrolyte, and at least one surfactant having an HLB of at least about 6 at 25° C. The polyelectrolytes may be branched and/or crosslinked, the compositions may include at least one of water and a water-soluble solvent, and the compositions may include an oily continuous phase.

### CROSS-REFERENCE TO RELATED APPLICATIONS

This non provisional application claims the benefit of French Application No. 04 53120 filed on Dec. 21, 2004 and U.S. Provisional Application No. 60/642,992 filed on Jan. 12, 2005.

### BACKGROUND

The present invention relates to cosmetic compositions for making up and/or for the nontherapeutic care of keratin materials, for example the skin, the lips and/or keratin fibers, which are capable of resisting water and of being easily removed.

For example, the compositions according to the invention may constitute a makeup product for the face, the body and/or the lips and for keratin fibers, for instance the eyelashes, the eyebrows and the hair, and for example an eyelash makeup product.

It may be for example a makeup composition, a transparent or colored composition to be applied over or under a makeup, also known, respectively, as “top coat” or “base coat”, or alternatively a composition for treating the eyelashes.

The composition according to the invention may be in the form of a product for the eyelashes or mascara, of a product for the eyebrows or of a makeup product for the hair. It may be for example a mascara.

There are in practice essentially two types of mascara formulation, namely, firstly, mascaras with an aqueous continuous phase, known as “emulsion mascaras”, which are in the form of an emulsion of waxes in water, and, secondly, mascaras with a solvent or oil continuous phase, which are anhydrous or have a low content of water and/or water-soluble solvents, known as “waterproof mascaras”, which are formulated in the form of a dispersion, in nonaqueous solvents, of at least one oily structuring agent which may be a wax, a polymer, in particular a semi-crystalline polymer, or a lipophilic gelling agent.

The present invention relates for example to the compositions or mascaras of “waterproof” type as defined above.

These “waterproof” mascaras are known to give rise to difficult, or even impossible, makeup removal with certain makeup removers, for example makeup removers that are mainly water-based or water-soluble, for example aqueous solutions. The makeup removal is therefore as a general rule performed using specific makeup removers based on oils or on organic solvents. However, these makeup removers may be irritant to the eyes, may especially cause stinging or may leave a veil on the eyes, or alternatively may leave an uncomfortable greasy residual film on the skin around the eyes (eyelids).

There is therefore a need for cosmetic makeup compositions that are capable of showing both good water resistance and a good ability to be removed, including with the usual makeup removers.

Documents FR 2 785 801, EP 1 152 022, FR 2 774 996, WO 95/35089 and WO 99/26445 describe thickening compositions, known as “thickening latices”, “thickeners” or “inverse latices”.

Documents FR 2 785 801 and FR 2 774 996 especially disclose compositions comprising an aqueous phase, an oily phase, an emulsifier of O/W (oil-in-water) type and an emulsifier of W/O (water-in-oil) type, and also a branched or crosslinked anionic polyelectrolyte based on a monomer containing a strong acid function.

Document WO 99/52499 describes mainly lipsticks comprising a sodium polyacrylate, for the purpose of producing a volumizing effect.

### SUMMARY

It has now been discovered that it is possible to obtain cosmetic compositions for making up and/or for the nontherapeutic care of keratin materials, which are capable of combining good water resistance and easy makeup removal irrespective of the type of makeup remover.

According to one exemplary embodiment, the present invention relates to a cosmetic composition for making up keratin materials, said composition having an oily continuous phase and comprising at least one polyelectrolyte, at least one surfactant with an HLB of greater than or equal to 6 at 25° C., and at least one dyestuff, wherein the polyelectrolyte is branched and/or crosslinked and in that the content of water and/or of water-soluble solvent optionally present is less than 50% by weight relative to the total weight of the composition.

The compositions according to the invention may for example be leave-on cosmetic compositions. Thus, the compositions according to the invention are generally different from the makeup-removing solutions that require, after use, subsequent rinsing aimed at completely removing them from the keratin material in question.

The present invention relates for example to compositions intended to be applied to keratin materials, and for example to keratin fibers.

Thus according to another exemplary embodiment, the present invention relates to a leave-on cosmetic composition for making up and/or caring for keratin fibers, said composition having an oily continuous phase and comprising at least one polyelectrolyte and at least one surfactant with an HLB of greater than or equal to 6 at 25° C., wherein the polyelectrolyte is branched and/or crosslinked and in that the content of water and/or of water-soluble solvent optionally present is less than 50% by weight relative to the total weight of the composition.

According to another exemplary embodiment, the present invention relates to a leave-on cosmetic composition for making up and/or for the nontherapeutic care of keratin fibers, said composition having an oily continuous phase and comprising at least one polyelectrolyte and at least one surfactant with an HLB of greater than or equal to 6 at 25° C., wherein the polyelectrolyte is chosen from an acrylamide/2-methyl-2-[(1-oxo-2-propenyl)amino]-1-propanesulphonic acid copolymer, a crosslinked starch glycolate in powdered form, a polyacrylate, a starch-based grafted copolymer, ionizable polysaccharide derivatives, polyacrylic acids, polyacrylic acid alkyl acrylate copolymers, AMPS (polyacrylamidomethylpropanesulphonic acid partially neutralized with aqueous ammonia and highly crosslinked), polyoxyethylenated AMPS/alkyl methacrylate copolymers (crosslinked or non-crosslinked), and a mixture of the latter, and preferably from an acrylamide/2-methyl-2-[(1-oxo-2-propenyl)amino]-1-propanesulphonic acid copolymer, a polyacrylate, and copolymers thereof.

According to another exemplary embodiment, the present invention relates to a cosmetic process for making up keratin materials, comprising at least one step of applying to the keratin materials a composition as defined above.

According to another exemplary embodiment, the present invention relates to a cosmetic process for making up and/or for the nontherapeutic care of keratin fibers, comprising at least one step of applying to the keratin fibers a leave-on composition as defined above.

According to another exemplary embodiment, a subject of the present invention is a made-up support comprising a makeup that can be obtained according to any one of the processes as defined above. In exemplary embodiments, a made-up support includes a makeup provided on a support, the makeup include a composition according to the present invention.

According to another exemplary embodiment, the present invention relates to a method for preparing a cosmetic composition for making up keratin materials, that has an oily continuous phase, and that exhibits good water resistance and is easily removed comprising the use of the combination of at least one polyelectrolyte and of at least one surfactant with an HLB of greater than or equal to 6 at 25° C.

According to another exemplary embodiment, the present invention relates to a method for preparing a leave-on cosmetic composition for making up and/or for the nontherapeutic care of keratin fibers, that has an oily continuous phase, and that exhibits good water resistance and is easily removed comprising the use of the combination of at least one polyelectrolyte and of at least one surfactant with an HLB of greater than or equal to 6 at 25° C.

As emerges from the examples below, the compositions according to the invention show such technical advantages.

### DETAILED DESCRIPTION OF EMBODIMENTS

The expression “cosmetic composition that has an oily continuous phase” is intended to mean a system capable of becoming diluted or dispersed on contact with said solvent medium or oil.

The expression “volatile organic oil or solvent” is intended to mean an organic oil or solvent (or non-aqueous medium) capable of evaporating on contact with the skin in less than one hour, at ambient temperature and atmospheric pressure. The volatile oil is a volatile cosmetic oil that is liquid at ambient temperature, for example having a non-zero vapour pressure at ambient temperature and atmospheric pressure, for example having a vapour pressure ranging from 0.13 Pa to 40 000 Pa (10⁻³ to 300 mmHg), and for example ranging from 1.3 Pa to 8000 Pa (0.01 to 60 mmHg).

In the context of the present invention, the term “keratin materials” includes the skin, the lips, the nails, the hair, the eyelashes and the eyebrows.

In the context of the present invention, the term “keratin fibers” is intended to mean for example the hair, the eyelashes and the eyebrows. Furthermore, the making up of the skin includes for example the making up of the body, the hands, the neck or the face.

The composition according to the invention comprises a physiologically acceptable medium, for example a cosmetically acceptable medium, i.e. a medium that is for example compatible with keratin materials and for example keratin fibers such as the hair, the eyelashes and the eyebrows.

In the context of the present invention, the term “cosmetically acceptable” is intended to mean a compound whose use is compatible with application to the keratin materials.

In general, for the sake of simplicity, and unless otherwise mentioned, the contents are indicated as solids.

Polyelectrolyte

The term “polyelectrolyte” is intended to mean a macromolecular substance that has the capacity of dissociating when it is dissolved in water or in any other ionizing medium, to give at least one ion. In other words, a polyelectrolyte is a polymer comprising at least one ionizable monomer.

The polyelectrolyte may give polyions, for example polyanions, when it is dissociated in water. A polyelectrolyte may be a polyacid, a polybase, a polysalt or a polyampholyte. In the context of the invention, it is for example a polyacid, and for example a strong polyacid.

The polyelectrolyte included in the cosmetic compositions according to the present invention may be a branched and/or crosslinked anionic polymer.

The polyelectrolyte may also be capable of forming a gel in an aqueous solution, beyond a concentration of greater than or equal to 0.1% by weight of solids, for example ≧0.3% by weight, relative to the total weight of the composition. This gel can be characterized, by oscillating rheology (ν=1 Hz), by means of a flow threshold τ_{c} at least equal to 10 Pa.

Furthermore, when the cosmetic composition according to the present invention comprises a film-forming polymer, the polyelectrolyte may differ from this film-forming polymer.

The counterions of the polyions formed during the dissociation may be inorganic or organic, of any nature.

When the polyelectrolyte is a branched or crosslinked anionic polymer, the cations may be alkali metal or alkaline earth metal cations such as sodium or potassium, or alternatively the ammonium ion.

The sodium cation Na⁺ may for example be used, which is why it is mainly cited in the list of polyelectrolytes that follows, without this constituting any limitation to this specific counterion.

As a polyelectrolyte, mention may be made of:

the acrylamide/Na AMPS copolymer such as Simulgel 600® in emulsion form containing polysorbate 80 as surfactant and containing isohexadecane as oil phase, sold by the company Seppic, or alternatively Simulgel EG®, Simulgel A® and Simulgel 501® sold by the same company.

Simulgel 600® is for example described in document FR 2 785 801. It is in reality an inverse latex. The AMPS polyelectrolyte is 2-methyl-2-[(1-oxo-2-propenyl)amino]-1-propanesulphonic acid partially or totally salified especially in sodium salt or ammonium salt form to a proportion of 30 to 50 mol % in the mixture comprising AMPS and also an acrylamide, which is itself present in a proportion of 50 to 70%;

crosslinked sodium starch glycolate in powder form;

sodium polyacrylates such as Norsocryl S35® sold by the company Atofina, or Cosmedia SP® sold by the company Cognis;

ionizable polysaccharide derivatives such as cellulose salts and sodium alginates;

starch-based grafted copolymers such as Waterlock® (A-180 and G-400, for example) from Grain Processing Corporation;

polyacrylic acids of Synthalen K® type;

polyacrylic acid alkyl acrylate copolymers of Pemulen® type;

AMPS (polyacrylamidomethylpropanesulphonic acid partially neutralized with aqueous ammonia and highly crosslinked), for example sold by the company Clariant;

polyoxyethylenated AMPS/alkyl methacrylate copolymers (crosslinked or non-crosslinked);

sodium carboxymethylcellulose and any ionizable cellulose derivatives, and

mixtures thereof.

Sodium polyacrylate and acrylamide/AMPS copolymer and copolymers thereof may for example be suitable for the invention.

It will of course be arranged for the polyelectrolyte content to be adjusted such that the removability is effectively improved while at the same time not being detrimental to the water resistance of the cosmetic composition.

It is understood that the amount of polyelectrolyte can vary significantly depending on the nature of the polyelectrolyte. In general, this amount is at least equal to the amount that is necessary and sufficient to give said composition better removability. It is also referred to as the effective amount.

This removability may for example be assessed by means of the test given in the examples below.

According to one exemplary embodiment, the polyelectrolyte is present at a content by weight, relative to the total weight of the composition, of greater than or equal to 0.05%, and for example greater than or equal to 0.1%, and for example greater than or equal to 0.5%.

According to one exemplary embodiment, the polyelectrolyte may be present in the cosmetic composition at a content ranging from 0.05 to 15% by weight, for example from 0.1 to 10% by weight, and for example from 0.5 to 5% by weight, relative to the total weight of the composition.

Without this constituting any limitation to the invention, the inventors have put forward the hypothesis that the polyelectrolyte in dispersion in the oil acts as a “water pump”. Thus, this “water pump” role is more clearly seen when the composition is brought into contact with an aqueous phase. Because of the presence of the high osmotic pressure in the aqueous reservoirs containing the polyelectrolyte due to the presence of counterions, said reservoirs swell until phase inversion is obtained.

Thus, at the macroscopic level, during the makeup removal, the film of the cosmetic composition according to the invention hydrates by means of the numerous “water pumps” microdispersed in the film. Said film becomes brittle and brings about its mechanical rupture. Fragmentation of the film then takes place.

The presence of the surfactant with an HLB of greater than or equal to 6 at 25° C. makes it possible to ultimately stabilize the cosmetic composition once hydrated, i.e. in the form of an emulsion having an aqueous continuous phase, in other words in the form of an oil-in-water emulsion.

Surfactant

The composition according to the invention may contain at least one surfactant with an HLB of greater than or equal to 6 at 25° C.

According to one exemplary embodiment, it or they may be present at a content by weight of greater than or equal to 0.1% relative to the total weight of the composition. It (they) may be present for example in a proportion ranging from 0.1 to 30%, for example from 0.5 to 15%, and for example from 1.5 to 10% by weight, relative to the total weight of the composition.

It is understood that, when the polyelectrolyte is incorporated into the composition of the invention in the form of a composition already formulated with a surfactant with an HLB of greater than or equal to 6 at 25° C., the amount of surfactant defined above takes into account the amount of said surfactant included in the polyelectrolyte formulation.

The expression “HLB of greater than or equal to 6” is intended to mean a surfactant having at 25° C. an HLB (hydrophilic-lipophilic balance), within the Griffin meaning, of greater than or equal to 6.

The HLB value according to Griffin is defined in J. Soc. Cosm. Chem. 1954 (volume 5), pages 249-256.

Reference may be made to the document “Encyclopedia of Chemical Technology, Kirk-Othmer”, volume 22, p. 333-432, 3rd edition, 1979, Wiley, for the definition of the (emulsifying) properties and functions of surfactants, in particular p. 347-377 of this reference, for the nonionic surfactants.

The surfactant with an HLB of greater than or equal to 6 at 25° C. included in the cosmetic composition according to the present invention may be ionic, nonionic or of mixed ionic and nonionic nature.

Among the nonionic surfactants with an HLB of greater than or equal to 6 at 25° C. that may be present in the compositions according to the present invention, used alone or as a mixture, mention may for example be made of:

oxyethylenated and/or oxypropylenated ethers (which may comprise from 1 to 150 oxyethylene and/or oxypropylene group) of glycerol;

oxyethylenated and/or oxypropylenated ethers (which may comprise from 1 to 150 oxyethylene and/or oxypropylene groups) of fatty alcohols (for example of C₈-C₂₄ and for example C₁₂-C₁₈ alcohol), such as the oxyethylenated cetearyl alcohol ether containing 30 oxyethylene groups (CTFA name “Ceteareth-30”) and the oxyethylenated ether of the mixture of C₁₂-C₁₅ fatty alcohols comprising 7 oxyethylene groups (CTFA name “C12-15 Pareth-7”), for example sold under the name Neodol 25-7® by Shell Chemicals;

fatty acid esters (for example of a C₈-C₂₄ acid and for example C₁₆-C₂₂ acid) of polyethylene glycol (which may comprise from 1 to 150 ethylene glycol units), such as PEG-50 stearate and PEG-40 monostearate, for example sold under the name Myrj 52P® by the company ICI Uniquema;

oxyethylenated and/or oxypropylenated fatty acid esters (for example of a C₈-C₂₄ and for example C₁₆-C₂₂ acid) of glycerol ethers (which may comprise from 1 to 150 oxyethylene and/or oxypropylene groups), for instance PEG-200 glyceryl monostearate sold under the name Simulsol 220 TM® by the company Seppic; glyceryl stearate polyethoxylated with 30 ethylene oxide groups, for instance the product Tagat S® sold by the company Goldschmidt, glyceryl oleate polyethoxylated with 30 ethylene oxide groups, for instance the product Tagat O® sold by the company Goldschmidt, glyceryl cocoate polyethoxylated with 30 ethylene oxide groups, for instance the product Varionic LI 13® sold by the company Sherex, glyceryl isostearate polyethoxylated with 30 ethylene oxide groups, for instance the product Tagat L® sold by the company Goldschmidt, and glyceryl laurate polyethoxylated with 30 ethylene oxide groups, for instance the product Tagat I® from the company Goldschmidt;

oxyethylenated and/or oxypropylenated fatty acid esters (for example of a C₈-C₂₄ and for example C₁₆-C₂₂ acid) of sorbitol ethers (which may comprise from 1 to 150 oxyethylene and/or oxypropylene groups), for instance polysorbate 60 sold under the name Tween 60® by the company Uniquema, and also polysorbate 80, polysorbate 40 and polysorbate 20;

dimethicone copolyol, such as that sold under the name Q2-5220® by the company Dow Corning;

dimethicone copolyol benzoate (Finsolv SLB 101® and 201® from the company Fintex);

copolymers of propylene oxide and of ethylene oxide, also known as EO/PO polycondensates;

and mixtures thereof.

The EO/PO polycondensates are for example copolymers consisting of polyethylene glycol and polypropylene glycol blocks, for instance polyethylene glycol/polypropylene glycol/polyethylene glycol triblock polycondensates. These triblock polycondensates have, for example, the following chemical structure:
H—(O—CH₂—CH₂)ₐ—(O—CH(CH₃)—CH₂)_{b}—(O—CH₂—CH₂)ₐ—OH,
in which formula a ranges from 2 to 120, and b ranges from 1 to 100.

The EO/PO polycondensate may for example have a weight-average molecular weight ranging from 1000 to 15 000, and for example ranging from 2000 to 13 000. In one exemplary embodiment, said EO/PO polycondensate has a cloud point, at 10 g/l in distilled water, of greater than or equal to 20° C., for example greater than or equal to 60° C. The cloud point is measured according to ISO standard 1065. As EO/PO polycondensates that may be used according to the invention, mention may be made of the polyethylene glycol/polypropylene glycol/polyethylene glycol triblock polycondensates sold, for example, under the names Synperonic®, for instance Synperonic PE/L44® and Synperonic PE/F127® by the company ICI.

Among the ionic surfactants, which may be anionic or cationic, with an HLB of greater than or equal to 6 at 25° C., which may be present in the composition according to the present invention, used alone or as a mixture, mention may for example be made of:

silicone surfactants, for instance dimethicone copolyol phosphates such as the product sold under the name Pecosil PS 100® by the company Phoenix Chemical,

amino acid derivatives, such as lauryl sarcosinate and lauryl taurate,

C₁₆-C₃₀ fatty acid salts, for example those derived from amines, for instance triethanolamine stearate,

polyoxyethylenated fatty acid salts, for example those derived from amines or alkali metal salts, and mixtures thereof,

phosphoric esters and salts thereof, such as “DEA oleth-10 phosphate” (Crofados N 10N® from the company Croda),

sulphosuccinates such as “Disodium PEG-5 citrate lauryl sulphosuccinate” and “Disodium ricinoleamido MEA sulphosuccinate”,

alkyl ether sulphates, such as sodium lauryl ether sulphate,

isethionates,

acylglutamates such as “Disodium hydrogenated tallow glutamate” (Amisoft HS-21 R® sold by the company Ajinomoto), and mixtures thereof.

Triethanolamine stearate is most particularly suitable for the invention. The latter is generally obtained by simple mixing of stearic acid and triethanolamine.

Representative cationic surfactants that may for example be mentioned include:

alkylimidazolidiniums, such as isostearylethylimidonium ethosulphate,

ammonium salts, such as N,N,N,-trimethyl-1-docosanamimium chloride (behentrimonium chloride).

The compositions according to the invention may also contain one or more amphoteric surfactants, for instance N-acylamino acids, such as N-alkylaminoacetates and cocoamphodiacetate.

Care will of course be taken to make sure that the surfactant with an HLB of greater than or equal to 6 at 25° C. is present in an amount such that the composition is indeed in the form of an emulsion that has an oily continuous phase.

The nonionic and ionic surfactants with an HLB of greater than or equal to 6 at 25° C. described above can also be present in combination.

Moreover, the cosmetic compositions according to the present invention may comprise one or more surfactant(s) with an HLB of greater than or equal to 10 at 25° C., and for example with an HLB of greater than or equal to 12.

The composition of the invention, in so far as it is a composition that has an oily continuous phase, may also contain a surfactant with a low HLB, i.e. less than 6, among which mention may be made of:

nonionic surfactants with an HLB of less than 6 at 25° C.:

esters and ethers of saccharides, such as sucrose stearate, sucrose cocoate, sorbitan stearate and mixtures thereof, for instance Arlatone 2121® sold by the company ICI;

fatty acid esters (for example of a C₈-C₂₄ and for example C₁₆-C₂₂ acid) of a polyol, for example of glycerol or of sorbitol, such as glyceryl stearate such as the product sold under the name Tegin M® by the company Goldschmidt, glyceryl laurate such as the product sold under the name lmwitor 312® by the company Huls, polyglyceryl-2 stearate, sorbitan tristearate or glyceryl ricinoleate;

the mixture of cyclomethicone/dimethicone copolyol sold under the name Q2-3225C® by the company Dow Corning;

fatty alcohols such as cetyl alcohol or stearyl alcohol;

ethoxylated fatty alcohols with an ethoxylation number such that the HLB is less than 6.

Fatty acid esters of oxyethylenated and/or oxypropylenated sorbitol ethers (which may comprise from 1 to 150 oxyethylene groups), such as polysorbate 20 with an HLB of 16.7, polysorbate 40 with an HLB of 15.6, polysorbate 60 with an HLB of 14.9 and polysorbate 80 with an HLB of 15.0, are for example suitable for the invention as surfactants with an HLB of greater than or equal to 6 at 25° C.

The composition according to the invention may comprise other surfactant(s) which is (are), for example, introduced into the composition through the introduction of the aqueous dispersion of particles of a polymer, these surfactants being those conventionally used in order to stabilize them.

Oils

The composition according to the invention may comprise at least one oil or one organic solvent. It may be a mixture of oils or of organic solvents.

The composition according to the invention may comprise a total amount of oil ranging from 10 to 90%, for example from 15 to 80%, and for example from 20 to 60% by weight, relative to the total weight of the composition, which can also be referred to as nonaqueous solvent medium.

The oil(s) present in the composition of the invention may be chosen from volatile oils and/or non-volatile oils, and mixtures thereof.

In the context of the present invention, the cosmetic compositions may comprise for example volatile oils.

The expression “volatile oil or organic solvent” is intended to mean an oil or organic solvent (or nonaqueous medium) capable of evaporating on contact with the skin in less than one hour, at ambient temperature and atmospheric pressure.

The volatile oil is a volatile cosmetic oil that is liquid at ambient temperature and that for example has a non-zero vapour pressure at ambient temperature and atmospheric pressure, for example that has a pressure with a value ranging from 0.13 Pa to 40 000 Pa (10⁻³ to 300 mmHg), and for example ranging from 1.3 Pa to 8000 Pa (0.01 to 60 mmHg).

The volatile oils (or organic solvents) may be hydrocarbon-based oils, silicone oils or fluoro oils, or mixtures thereof.

The term “hydrocarbon-based oil” is intended to mean an oil containing mainly hydrogen and carbon atoms and, optionally, oxygen, nitrogen, sulphur, phosphorus atoms. The volatile hydrocarbon-based oils may be chosen from hydrocarbon-based oils having from 8 to 16 carbon atoms, and for example C₈-C₁₆ branched alkanes, for instance C₈-C₁₆ isoalkanes of petroleum origin (also called isoparaffins), for instance isododecane (also called 2,2,4,4,6-pentamethylheptane), isodecane, isohexadecane, and, for example, the oils sold under the trade names “Isopars®” or “Permetyls®”, C₈-C₁₆ branched esters, isohexyl neopentanoate, and mixtures thereof. Other volatile hydrocarbon-based oils such as petroleum distillates, for example those sold under the name “Shell Solt®” by the company Shell, can also be used.

As volatile oils, use may also be made of volatile silicones, for instance volatile linear or cyclic silicone oils, for example those that have a viscosity ≦6 centistokes (6×10⁻⁶ m²/s) and that for example have from 3 to 6 silicon atoms, these silicones optionally comprising one or more alkyl or alkoxy groups having one or two carbon atoms. As volatile silicone oils that may be used in the invention, mention may for example be made of octamethylcyclotetrasiloxane, decamethylcyclopentasiloxane, dodecamethylcyclohexa-siloxane, heptamethylhexyltrisiloxane, heptamethyloctyltrisiloxane, hexamethyldisiloxane, octamethyltrisiloxane, decamethyltetrasiloxane, dodecamethylpentasiloxane, heptamethylethyltrisiloxane, heptamethylbutyltrisiloxane, and mixtures thereof.

Use may also be made of volatile organic solvents that are for example fluorinated, such as nonafluoromethoxybutane or perfluoromethylcyclopentane.

In one exemplary embodiment, the volatile oil(s) may be chosen from hydrocarbon-based volatile oils having from 8 to 16 carbon atoms, such as isododecane, silicone volatile oils such as decamethylcyclopentasiloxane (D5), or dodecamethylcyclohexasiloxane (D6), and mixtures thereof.

The composition according to the invention may also comprise at least one non-volatile, water-insoluble compound that is liquid at ambient temperature, for example at least one non-volatile oil or organic solvent, which may for example be chosen from non-volatile hydrocarbon-based and/or silicone and/or fluoro oils.

As non-volatile hydrocarbon-based oils, mention may for example be made of:

hydrocarbon-based oils of plant origin, such as triglycerides consisting of fatty acid esters of glycerol, the fatty acids of which may have varied chain lengths from C₄ to C₂₄, it being possible for the latter to be linear or branched, and saturated or unsaturated; these oils are for example wheatgerm oil, sunflower oil, grapeseed oil, sesame oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin oil, marrow oil, blackcurrant oil, evening primrose oil, millet oil, barley oil, quinoa oil, rye oil, safflower oil, candlenut oil, passion flower oil or musk rose oil; or alternatively caprylic/capric acid triglycerides, for instance those sold by the company Stéarineries Dubois or those sold under the names Miglyol 810®, 812® and 818® by the company Dynamit Nobel;

synthetic ethers having from 10 to 40 carbon atoms;

linear or branched hydrocarbons of mineral or synthetic origin such as petroleum jelly, polydecenes, hydrogenated polyisobutene such as parleam, squalane, and mixtures thereof;

synthetic esters, for instance oils of formula R₁COOR₂ in which R₁ represents a linear or branched fatty acid residue containing from 1 to 40 carbon atoms and R₂ represents a hydrocarbon-based chain that is for example branched, containing from 1 to 40 carbon atoms provided that R₁+R₂ is ≧10, for instance Purcellin oil (cetostearyl octanoate), isopropyl myristate, isopropyl palmitate, C₁₂ to C₁₅ alcohol benzoates, hexyl laurate, diisopropyl adipate, isononyl isononanoate, 2-ethylhexyl palmitate, isostearyl isostearate, alcohol or polyalcohol octanoates, decanoates or ricinoleates, for instance propylene glycol dioctanoate; hydroxylated esters, for instance isostearyl lactate, diisostearyl malate; and pentaerythritol esters;

fatty alcohols that are liquid at ambient temperature, with a branched and/or unsaturated carbon-based chain having from 12 to 26 carbon atoms, for instance octyldodecanol, isostearyl alcohol, oleyl alcohol, 2-hexyldecanol, 2-butyloctanol and 2-undecylpentadecanol;

higher fatty acids such as oleic acid, linoleic acid or linolenic acid;

and mixtures thereof.

The non-volatile silicone oils that can be used in the composition according to the invention may be non-volatile polydimethylsiloxanes (PDMS), polydimethylsiloxanes comprising alkyl or alkoxy groups that are pendant and/or at the end of a silicone chain, these groups each having from 2 to 24 carbon atoms, phenyl silicones, for instance phenyl trimethicones, phenyl dimethicones, phenyl trimethylsiloxy diphenylsiloxanes, diphenyl dimethicones, diphenyl methyldiphenyltrisiloxanes and 2-phenylethyl trimethylsiloxysilicates.

The fluoro oils that can be used in the composition of the invention may for example be fluorosilicone oils, fluorinated polyethers or fluorinated silicones as described in document EP-A-847752.

The content of volatile oil or organic solvent in a composition according to the invention may range from 20 to 80% by weight, for example from 30 to 70% by weight, and for example from 35 to 60% by weight, relative to the total weight of the composition.

The content of non-volatile oil or organic solvent in a composition according to the invention may range from 0.01 to 30% by weight, for example from 0.1 to 25% by weight, and for example from 0.1 to 20% by weight, relative to the total weight of the composition.

Water and/or Water-Soluble Solvent

The compositions according to the invention may comprise an aqueous phase comprising water and/or at least one water-soluble solvent. In this case, the content of water and/or of water-soluble solvent may be less than or equal to 50% by weight, for example less than or equal to 40% by weight, for example less than or equal to 30% by weight, relative to the total weight of the composition.

In one exemplary embodiment, its content is less than 20% by weight, and it can range from 0.1 to 20%, and for example from 1 to 10% by weight, relative to the total weight of the composition.

In any event, the content of water and/or of water-soluble solvent(s) in the composition according to the invention is such that the composition remains in the form of an oily continuous phase, without phase inversion.

In the present invention, the term “water-soluble solvent” denotes a compound that is liquid at ambient temperature and water-miscible (miscibility in water of greater than 50% by weight at 25° C. and atmospheric pressure).

The water-soluble solvents that may be used in the compositions according to the invention may also be volatile.

Among the water-soluble solvents that may be used in the compositions according to the invention, mention may for example be made of lower monoalcohols having from 1 to 5 carbon atoms, such as ethanol and isopropanol, glycols having from 2 to 8 carbon atoms, such as ethylene glycol, propylene glycol, 1,3-butylene glycol and dipropylene glycol, C₃ and C₄ ketones and C₂-C₄ aldehydes.

The aqueous phase (water and/or water-soluble solvent(s)) may be introduced in unmodified form into the formulation according to the invention or may be incorporated therein by means of one or more ingredients constituting said composition. Thus, water may for example be introduced into the composition by means of the introduction of latex or of pseudolatex, i.e. of an aqueous dispersion of polymer particles.

Reference is made to an “anhydrous” composition when the content of water and/or water-soluble solvent(s) in the composition is less than 5% by weight relative to the total weight of the composition. This type of composition is also part of the invention.

All the components described below, which may be included in the composition according to the present invention, will of course be incorporated at contents such that their presence does not or does not substantially affect the desired effect, namely the maintaining of good water resistance while at the same time demonstrating an improved removability.

Structuring Agent

The composition according to the invention may comprise at least one agent for structuring the oily phase or organic solvent, chosen from waxes, semi-crystalline polymers and lipophilic gelling agents, and mixtures thereof.

The structuring agent may represent from 0.1 to 80% by weight relative to the total weight of the composition, for example from 0.5 to 50%, and for example from 1 to 40% by weight. The amount of oily structuring agent may be adjusted by those skilled in the art as a function of the structuring properties of said agents.

Wax(es)

The wax under consideration in the context of the present invention may be a lipophilic compound that is solid at ambient temperature (25° C.), with a solid/liquid reversible change of state, having a melting point of greater than or equal to 30° C., which may be up to 200° C. and for example up to 120° C.

By bringing the wax to the liquid form (melting), it is possible to make it miscible with the oils and to form a microscopically uniform mixture, but on cooling the mixture to ambient temperature, recrystallization of the wax in the oils of the mixture is obtained.

In particular, the waxes that are suitable for the invention may have a melting point of greater than or equal to 45° C., and for example greater than or equal to 55° C.

For the purpose of the invention, the melting point corresponds to the temperature of the most endothermic peak observed by thermal analysis (DSC) as described in ISO standard 11357-3; 1999. The melting point of the wax may be measured using a differentially scanning calorimeter (DSC), for example the calorimeter sold under the name “MDSC 2920” by the company TA Instruments.

The measuring protocol is as follows:

A sample of 5 mg of wax placed in a crucible is subjected to a first temperature rise ranging from −20° C. to 100° C., at a heating rate of 10° C./minute, it is then cooled from 100° C. to −20° C. at a cooling rate of 10° C./minute and is finally subjected to a second temperature rise ranging from −20° C. to 100° C. at a heating rate of 5° C./minute. During the second temperature rise, the variation in the difference in power absorbed by the empty crucible and by the crucible containing the sample of wax is measured as a function of the temperature. The melting point of the compound is the temperature value corresponding to the top of the peak of the curve representing the variation in the difference in absorbed power as a function of the temperature.

The waxes that may be used in the compositions according to the invention are chosen from waxes that are solid at ambient temperature of animal, plant, mineral or synthetic origin and mixtures thereof.

The waxes that may be used in the compositions according to the invention may have a hardness ranging from 0.01 MPa to 15 MPa, for example greater than 0.05 MPa, and for example greater than 0.1 MPa.

The hardness is determined by measuring the compression force, measured at 20° C. using a texturometer sold under the name TA-TX2i® by the company Rheo, equipped with a stainless steel cylindrical spindle of 2 mm in diameter, by measuring the change in force (compression force or stretching force) (F) as a function of time, during the following operation:

The spindle is displaced at a speed of 0.1 mm/s and then penetrates the wax to a penetration depth of 0.3 mm. When the spindle has penetrated the wax to a depth of 0.3 mm, the spindle is held still for 1 second (corresponding to the relaxation time) and is then withdrawn at a speed of 0.1 mm/s. During the relaxation time, the force (compression force) greatly decreases until it becomes zero, and then, during the withdrawal of the spindle, the force (stretching force) becomes negative and then rises again towards the value 0. The hardness corresponds to the maximum compression force measured between the surface of the spindle and the wax at the moment they come into contact. The value of this force is expressed in MPa.

To measure the hardness, the wax is melted at a temperature equal to the melting point of the wax +20° C. The molten wax is poured into a container 30 mm in diameter and 20 mm deep. The wax is recrystallized at ambient temperature (25° C.) for 24 hours and is then stored for at least 1 hour at 20° C., before performing the hardness measurement.

By way of illustration of the waxes that are suitable for the invention, mention may for example be made of hydrocarbon-based waxes, for instance beeswax, lanolin wax, Chinese insect waxes, sumach wax, paraffins, certain polyethylene waxes and waxy copolymers, and also esters thereof.

Mention may also be made of waxes obtained by catalytic hydrogenation of animal or plant oils having linear or branched C₈-C₃₂ fatty chains. Among these, mention may for example be made of isomerized jojoba oil such as the trans-isomerized partially hydrogenated jojoba oil manufactured or sold by the company Desert Whale under the commercial reference Iso-Jojoba-50®, hydrogenated sunflower oil, hydrogenated castor oil, hydrogenated coconut oil, hydrogenated lanolin oil and bis(1,1,1-trimethylolpropane) tetrastearate sold under the name Hest 2T-4S® by the company Heterene.

Mention may also be made of silicone waxes and fluorowaxes.

The waxes obtained by hydrogenation of castor oil esterified with cetyl alcohol, sold under the names Phytowax ricin 16L64® and 22L73® by the company Sophim may also be used. Such waxes are described in application FR-A-2792190.

According to one exemplary embodiment, the compositions according to the invention may comprise at least one wax referred to as a “tacky wax”, i.e. a wax with a tack of greater than or equal to 0.7 N.s and a hardness of less than or equal to 3.5 MPa.

The use of a tacky wax may for example allow the production of a cosmetic composition that applies easily to keratin fibers, with good attachment to the keratin fibers, and which leads to the formation of a smooth, homogeneous and thickening makeup.

The tacky wax used may for example have a tack ranging from 0.7 N.s to 30 N.s, for example greater than or equal to 1 N.s, for example ranging from 1 N.s to 20 N.s, for example greater than or equal to 2 N.s, for example ranging from 2 N.s to 10 N.s, and for example ranging from 2 N.s to 5 N.s.

The tack of the wax is determined by measuring the change in force (compression force or stretching force) as a function of time, at 20° C., using the texturometer sold under the name TA-TX2i® by the company Rheo, equipped with a conical acrylic polymer spindle forming an angle of 45°.

The measuring protocol is as follows:

The wax is melted at a temperature equal to the melting point of the wax +10° C. The molten wax is poured into a container 25 mm in diameter and 20 mm deep. The wax is recrystallized at ambient temperature (25° C.) for 24 hours such that the surface of the wax is flat and smooth, and the wax is then stored for at least 1 hour at 20° C. before measuring the tack.

The texturometer spindle is displaced at a speed of 0.5 mm/s, then penetrates the wax to a penetration depth of 2 mm. When the spindle has penetrated the wax to a depth of 2 mm, the spindle is held still for 1 second (corresponding to the relaxation time) and is then withdrawn at a speed of 0.5 mm/s.

During the relaxation time, the force (compression force) greatly decreases until it becomes zero, and then, during the withdrawal of the spindle, the force (stretching force) becomes negative and then rises again towards the value 0. The tack corresponds to the integral of the curve of the force as a function of time for the part of the curve corresponding to the negative values of the force (stretching force). The tack value is expressed in N.s.

The tacky wax that may be used may be a hardness of less than or equal to 3.5 MPa, for example ranging from 0.01 MPa to 3.5 MPa, for example ranging from 0.05 MPa to 3 MPa, or for example ranging from 0.1 MPa to 2.5 MPa.

The hardness is measured according to the protocol described previously.

Tacky waxes that may be used include a C₂₀-C₄₀ alkyl (hydroxystearyloxy)stearate (the alkyl group containing from 20 to 40 carbon atoms), alone or as a mixture, for example a C₂₀-C₄₀ alkyl 12-(12′-hydroxystearyloxy)stearate, of formula (I): 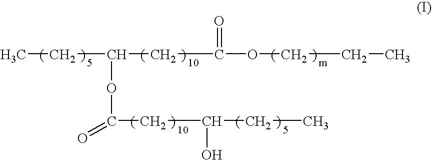
in which m is an integer ranging from 18 to 38, or a mixture of compounds of formula (I).

Such a wax is for example sold under the names Kester Wax K 82 P® and Kester Wax K 80 P® by the company Koster Keunen.

The waxes mentioned above may have a starting melting point of less than 45° C.

In the present invention, waxes provided in the form of small particles having a dimension expressed as the mean “effective” volume diameter D[4.3] of the order of 0.5 to 30 micrometres, for example from 1 to 20 micrometres, and for example from 5 to 10 micrometres, which are referred to hereinafter as “microwaxes”, may also be used. For the purposes of distinction, the waxes used according to the invention in the form of fragments of larger size are subsequently referred to as “conventional waxes”.

As microwaxes that may be used in the compositions according to the invention, mention may for example be made of carnauba microwaxes, such as the product sold under the name MicroCare 350® by the company Micro Powders, synthetic microwaxes, such as the product sold under the name MicroEase 114S® by the company Micro Powders, microwaxes consisting of a mixture of carnauba wax and polyethylene wax, such as the products sold under the names Micro Care 300® and 310® by the company Micro Powders, microwaxes consisting of a mixture of camauba wax and synthetic wax, such as the product sold under the name Micro Care 325® by the company Micro Powders, polyethylene microwaxes, such as the products sold under the names Micropoly 200®, 220®, 220L® and 250S® by the company Micro Powders, and polytetrafluoroethylene microwaxes, such as the products sold under the names Microslip 519® and 519 L® by the company Micro Powders.

In the composition according to the invention, it is of course possible to use a mixture of waxes, and for example to use one or more conventional waxes, such as, for example, a tacky wax and/or a wax with a starting melting point of greater than or equal to 45° C., and one or more waxes known as microwaxes. The composition according to the invention may comprise a content of waxes ranging from 0.1 to 70% by weight relative to the total weight of the composition; it may for example contain from 0.5 to 50%, for example from 1 to 30%, thereof.

Semi-Crystalline Polymers

The term “polymer” is intended to mean compounds containing two repeating units, for example at least three repeating units, and for example at least ten repeating units. The term “semi-crystalline polymer” is intended to mean polymers comprising a crystallizable portion, a crystallizable pendant chain or a crystallizable block in the backbone, and an amorphous portion in the backbone, and having a first-order reversible phase-change temperature, in particular of melting (solid-liquid transition). When the crystallizable portion is in the form of a crystallizable block of the polymeric backbone, the amorphous portion of the polymer is in the form of an amorphous block; in this case, the semi-crystalline polymer is a block copolymer, for example of the diblock, triblock or multiblock type, comprising at least one crystallizable block and at least one amorphous block. The term “block” may be intended to mean at least 5 identical repeating units. The crystallizable sequence(s) is (are) then of chemical nature different from the amorphous block(s).

The semi-crystalline polymer has a melting point of greater than or equal to 30° C. (for example ranging from 30° C. to 80° C.), for example ranging from 30° C. to 60° C. This melting point is a first-order change of state temperature.

This melting point may be measured by any known method and for example using a differential scanning calorimeter (DSC).

In one exemplary embodiment, the semi-crystalline polymer(s) to which the invention applies has (have) a number-average molecular mass of greater than or equal to 1000. In one exemplary embodiment, the semi-crystalline polymer(s) of the composition of the invention has (have) a number-average molecular mass \overline{M}n ranging from 2000 to 800 000, for example from 3000 to 500 000, for example from 4000 to 150 000, for example less than 100 000, and from 4000 to 99 000. They may have a number-average molecular mass of greater than 5600, ranging, for example, from 5700 to 99 000. For the purpose of the invention, the expression “crystallizable chain or block” is intended to mean a chain or block which, if it were alone, would reversibly change from the amorphous state to the crystalline state, depending on whether the system is above or below the melting point. For the purpose of the invention, a chain is a group of atoms which is pendant or lateral relative to the polymer backbone. A block is a group of atoms belonging to the backbone, this group constituting one of the repeating units of the polymer.

In one exemplary embodiment, the “crystallizable pendant chain” may be a chain containing at least 6 carbon atoms.

The semi-crystalline polymer may be chosen from block copolymers comprising at least one crystallizable block and at least one amorphous block, homopolymers and copolymers bearing at least one crystallizable side chain per repeating unit, and mixtures thereof.

Such polymers are described, for example, in document EP 1 396 259.

By way of specific example of a structuring semi-crystalline polymer that can be used in the composition according to the invention, mention may be made of the products Intelimer® from the company Landec described in the brochure “Intelimer® polymers”, Landec IP22 (Rev. 4-97). The polymers are in solid form at ambient temperature (25° C.) and bear crystallisable side chains.

Lipophilic Gelling Agents

The gelling agents that can be used in the compositions according to the invention may be organic or mineral, polymeric or molecular lipophilic gelling agents.

As mineral lipophilic gelling agents, mention may be made of optionally modified clays such as hectorites modified with a C₁₀ to C₂₂ fatty acid ammonium chloride, for instance the hectorite modified with distearyl dimethylammonium chloride such as, for example, the product sold under the name Bentone 38V® by the company Elementis.

Mention may also be made of fumed silica optionally subjected to a hydrophobic surface treatment, the particle size of which is less than 1 μm. Specifically, it is possible to chemically modify the surface of the silica, by chemical reaction generating a reduced number of silanol groups present at the surface of the silica. It is for example possible to substitute silanol groups with hydrophobic groups: a hydrophobic silica is then obtained. The hydrophobic groups may be:

trimethylsiloxyl groups, which may be obtained for example by treating fumed silica in the presence of hexamethyldisilazane. Silicas thus treated are known as “silica silylate” according to the CTFA (6th edition, 1995). They are, for example, sold under the references Aerosil R812® by the company Degussa, Cab-O-Sil TS-530® by the company Cabot;

dimethylsilyloxyl or polydimethylsiloxane groups, which may be obtained for example by treating fumed silica in the presence of polydimethylsiloxane or dimethyldichlorosilane. Silicas thus treated are known as “silica dimethyl silylate” according to the CTFA (6th edition, 1995). They are, for example, sold under the references Aerosil R972® and Aerosil R974® by the company Degussa, and Cab-O-Sil TS-610® and Cab-O-Sil TS-720® by the company Cabot.

The hydrophobic fumed silica may have for example a particle size that may be nanometric to micrometric, for example ranging from approximately 5 to 200 nm.

The polymeric organic lipophilic gelling agents are, for example, partially or totally crosslinked elastomeric organopolysiloxanes of three-dimensional structure, for instance those sold under the names KSG6®, KSG16® and KSG18® by the company Shin-Etsu, Trefil E-505C® and Trefil E-506C® by the company Dow-Corning, Gransil SR-CYC®, SR DMF10®, SR-DC556®, SR 5CYC gel®, SR DMF 10 gel® and SR DC 556 gel® by the company Grant Industries, and SF 1204® and JK 113® by the company General Electric; ethylcellulose, for instance the product sold under the name Ethocel® by the company Dow Chemical; polyamide-type polycondensates resulting from condensation between (α) at least one acid chosen from dicarboxylic acids containing at least 32 carbon atoms, such as dimer fatty acids, and (β) an alkylene diamine, and for example ethylenediamine, in which the polyamide polymer comprises at least one terminal carboxylic acid group esterified or amidated with at least one monoalcohol, or one monoamine containing from 12 to 30 linear and saturated carbon atoms, and for example ethylenediamine/stearyl dilinoleate copolymers such as the product sold under the name Uniclear 100 VG® by the company Arizona Chemical; galactommannans comprising from one to six, and for example from two to four, hydroxyl groups per saccharide, substituted with a saturated or unsaturated alkyl chain, for instance guar gum alkylated with C₁ to C₆, and for example C₁ to C₃, alkyl chains, and mixtures thereof. Block copolymers of “diblock”, “triblock” or “radial” type, of the polystyrene/polyisoprene or polystyrene/polybutadiene type, such as the products sold under the name Luvitol HSB® by the company BASF, of the polystyrene/copoly(ethylene-propylene) type, such as the product sold under the name Kraton® by the company Shell Chemical Co, or alternatively the polystyrene/copoly(ethylene-butylene) type, blends of triblock and radial (star) copolymers in isododecane, such as the product sold by the company Penreco under the name Versagel®, for instance the blend of butylene/ethylene/styrene triblock copolymer and of ethylene/propylene/styrene star copolymer in isododecane (Versagel M 5960).

Among the lipophilic gelling agents that can be used in the compositions according to the invention, mention may also be made of esters of dextrin and of a fatty acid, such as dextrin palmitates, especially such as the products sold under the names Rheopearl TL® or Rheopearl KL® by the company Chiba Flour.

Film-Forming Polymer

The composition according to the invention may comprise, according to one exemplary embodiment, at least one film-forming polymer.

The film-forming polymer may be present in the composition according to the invention at a solids content ranging from 0.1% to 60% by weight relative to the total weight of the composition, for example from 0.5% to 40% by weight, and for example from 1% to 30% by weight.

In the present invention, the term “film-forming polymer” is intended to mean a polymer that is capable of forming, by itself or in the presence of an auxiliary film-forming agent, a film that is macroscopically continuous and adheres to keratin fibers, and for example a cohesive film, and for example a film whose cohesion and mechanical properties are such that it is possible that said film can be isolated and handled in isolation, for example when said film is prepared by pouring onto an anti-adhesive surface such as a teflon or silicone surface.

Among the film-forming polymers that can be used in the composition of the present invention, mention may be made of synthetic polymers, of radical type or of polycondensate type, polymers of natural origin, and blends thereof.

These film-forming polymers may be for example different from the polyelectrolyte defined above.

Liposoluble Polymers

According to one exemplary embodiment, the film-forming polymer may be a polymer solubilized in a liquid fatty phase comprising oils or organic solvents such as those described above (the film-forming polymer is then referred to as a liposoluble polymer). For the purpose of the invention, the term “liquid fatty phase” is intended to mean a fatty phase that is liquid at ambient temperature (25° C.) and atmospheric pressure (760 mmHg, i.e. 10⁵ Pa), and that is composed of one or more fatty substances that are liquid at ambient temperature, such as the oils described above, which may be compatible with one another.

In one exemplary embodiment, the liquid fatty phase may comprise a volatile oil, optionally as a mixture with a non-volatile oil, it being possible for the oils to be chosen from the oils mentioned above.

By way of example of a liposoluble polymer, mention may be made of copolymers of vinyl ester (the vinyl group being directly connected to the oxygen atom of the ester group and the vinyl ester having a linear or branched, saturated hydrocarbon-based radical containing from 1 to 19 carbon atoms, linked to the carbonyl of the ester group) and of at least one other monomer, which may be a vinyl ester (other than the vinyl ester already present), an α olefin (having from 8 to 28 carbon atoms), an alkyl vinyl ether (in which the alkyl group contains from 2 to 18 carbon atoms), or an allyl or methallyl ester (having a linear or branched, saturated hydrocarbon-based radical containing from 1 to 19 carbon atoms, linked to the carbonyl of the ester group).

These copolymers may be crosslinked with crosslinking agents which may be either of the vinyl type, or of the allyl or methallyl type, such as tetraallyloxyethane, divinylbenzene, divinyl octanedioate, divinyl dodecanedioate, and divinyl octadecanedioate.

As examples of these copolymers, mention may be made of the copolymers: vinyl acetate/allyl stearate, vinyl acetate/vinyl laurate, vinyl acetate/vinyl stearate, vinyl acetate/octadecene, vinyl acetate/octadecyl vinyl ether, vinyl propionate/allyl laurate, vinyl propionate/vinyl laurate, vinyl stearate/octadecene-1, vinyl acetate/dodecene-1, vinyl stearate/ethyl vinyl ether, vinyl propionate/cetyl vinyl ether, vinyl stearate/allyl acetate, vinyl 2,2-dimethyloctanoate/vinyl laurate, allyl 2,2-dimethylpentanoate/vinyl laurate, vinyl dimethylpropionate/vinyl stearate, allyl dimethylpropionate/vinyl stearate, vinyl propionate/vinyl stearate, crosslinked with 0.2% of divinylbenzene, vinyl dimethylpropionate/vinyl laurate, crosslinked with 0.2% of divinylbenzene, vinyl acetate/octadecyl vinyl ether, crosslinked with 0.2% of tetraallyloxyethane, vinyl acetate/allyl stearate, crosslinked with 0.2% of divinylbenzene, vinyl acetate/octadecene-1, crosslinked with 0.2% of divinylbenzene, and allyl propionate/allyl stearate, crosslinked with 0.2% of divinylbenzene.

As liposoluble film-forming polymers, mention may also be made of liposoluble copolymers, and for example those resulting from the copolymerization of vinyl esters having from 9 to 22 carbon atoms or of alkyl acrylates or methacrylates, the alkyl radicals having from 10 to 20 carbon atoms.

Such liposoluble copolymers may be chosen from copolymers of poly(vinyl stearate), of poly(vinyl stearate) crosslinked with divinylbenzene, of diallyl ether or of diallyl phthalate, copolymers of poly(stearyl (meth)acrylate), of poly(vinyl laurate), of poly(lauryl(meth)acrylate), it being possible for these poly(meth)acrylates to be crosslinked with tetraethylene glycol or ethylene glycol dimethacrylate.

The liposoluble copolymers defined above are known and are for example described in application FR-A-2232303; they may have a weight-average molecular weight ranging from 2000 to 500 000, and for example from 4000 to 200 000.

As liposoluble film-forming polymers that can be used in the invention, mention may also be made of polyalkylenes, and for example copolymers of C₂-C₂₀ alkenes, for instance polybutene, alkylcelluloses with a C₁ to C₈ saturated or unsaturated, linear or branched alkyl radical, for instance ethylcellulose and propylcellulose, copolymers of vinylpyrrolidone (VP) and for example copolymers of vinylpyrrolidone and of C₂ to C₄₀, and for example C₃ to C₂₀, alkene. By way of example of a VP copolymer that can be used in the invention, mention may be made of the copolymer of VP/vinyl acetate, VP/ethyl methacrylate, butyl polyvinylpyrrolidone (PVP), VP/ethyl methacrylate/methacrylic acid, VP/eicosene, VP/hexadecene, VP/triacontene, VP/styrene, and VP/acrylic acid/lauryl methacrylate.

Mention may also be made of silicone resins, that may be soluble or can be swollen in silicone oils, which are crosslinked polyorganosiloxane polymers. The nomenclature of silicone resins is known as “MDTQ”, the resin being described as a function of the various siloxane monomer units that it comprises, each of the letters “MDTQ” characterizing a type of unit.

By way of examples of commercially available polymethylsilsesquioxane resins, mention may be made of those which are sold:

by the company Wacker under the reference Resin MK, such as Belsil PMS MK;

by the company Shin-Etsu under the reference KR-220L.

As siloxysilicate resins, mention may be made of trimethylsiloxysilicate (TMS) resins such as those sold under the reference SR1000 by the company General Electric or under the reference TMS 803 by the company Wacker. Mention may also be made of the trimethylsiloxysilicate resins sold in a solvent such as cyclomethicone, sold under the name “KF-7312J” by the company Shin-Etsu, or “DC 749” or “DC 593” by the company Dow Corning.

Silicone-based polyamides of the polyorganosiloxane type, such as those described in documents U.S. Pat. No. 5,874,069, U.S. Pat. No. 5,919,441, U.S. Pat. No. 6,051,216 and U.S. Pat. No. 5,981,680 can also be used.

These silicone polymers may belong to the following two families:

(1) polyorganosiloxanes comprising at least two groups capable of establishing hydrogen interactions, these two groups being located in the chain of the polymer, and/or

(2) polyorganosiloxanes comprising at least two groups capable of establishing hydrogen interactions, these two groups being located on grafts or branches.

According to one exemplary embodiment, the film-forming polymer is a film-forming linear block ethylenic polymer which may comprise at least a first block and at least a second block having different glass transition temperatures (Tg), said first and second blocks being connected to one another via an intermediate block comprising at least one monomer constituting the first block and at least one monomer constituting the second block.

In one exemplary embodiment, the first and second blocks of the block polymer may be incompatible with one another.

Such polymers are described, for example, in documents EP 1411069 or WO 04/028488.

Water-Soluble Polymer

According to another exemplary embodiment, the film-forming polymer may be a water-soluble polymer and may be present in an aqueous phase of the composition; the polymer is therefore solubilized in the aqueous phase of the composition. As examples of water-soluble film-forming polymers, mention may be made of:

proteins, for instance proteins of plant origin, such as wheat proteins or soybean proteins; proteins of animal origin, such as keratins, for example keratin hydrolysates and sulphonic keratins;

cellulose polymers, such as hydroxyethylcellulose, hydroxypropylcellulose, methylcellulose or ethylhydroxyethylcellulose;

acrylic polymers or copolymers, such as polyacrylates or polymethacrylates;

vinyl polymers, for instance polyvinylpyrrolidones, copolymers of methyl vinyl ether and of malic anhydride, copolymer of vinyl acetate and of crotonic acid, copolymers of vinylpyrrolidone and of vinyl acetate; copolymers of vinylpyrrolidone and of caprolactam; polyvinyl alcohol;

optionally modified polymers of natural origin, such as:

gum arabics, guar gum, xanthan derivatives, karaya gum;

alginates and carrageenans;

glycosaminoglycans, and hyaluronic acid and its derivatives;

shellac resin, sandarac gum, dammar resins, elemi gums and copal resins;

deoxyribonucleic acid;

muccopolysaccharides such as chondroitin sulphates,

and mixtures thereof.

Polymers of Natural Origin

The polymers of natural origin, which are optionally modified, may be chosen from shellac resin, sandarac gum, dammar resins, the elemi gums, copal resins and cellulose polymers, and mixtures thereof.

Polymers in Dispersed Form

The film-forming polymer may be present in the composition in the form of particles dispersed in an aqueous phase or in a nonaqtieous solvent phase, generally known generally known as latex or pseudolatex. The techniques for preparing these dispersions are well known to those skilled in the art.

a) Aqueous Dispersion

As an aqueous dispersion of a film-forming polymer, use may be made of the acrylic dispersions sold under the names Neocryl XK-90®, Neocryl A-1070®, Neocryl A-1090®, Neocryl BT-62®, Neocryl A-1079® and Neocryl A-523® by the company Avecia-Neoresins, Dow Latex 432® by the company Dow Chemical, Daitosol 5000 AD® or Daitosol 5000 SJ® by the company Daito Kasey Kogyo; Syntran 5760® by the company Interpolymer, Allianz Opt® by the company Rohm and Haas, or alternatively the aqueous dispersions of polyurethane sold under the names Neorez R-981® and Neorez R-974® by the company Avecia-Neoresins, Avalure UR-405®, Avalure UR-410®, Avalure UR-425®, Avalure UR-450®, Sancure 875®, Sancure 861®, Sancure 878® and Sancure 2060® by the company Goodrich, Impranil 85® by the company Bayer, Aquamere H-1511® by the company Hydromer; sulphopolyesters sold under the trade mark Eastman AQ® by the company Eastman Chemical Products, vinyl dispersions such as Mexomere PAM®, aqueous dispersions of polyvinyl acetate, for instance “Vinybrano” from the company Nisshin Chemical or the product sold by the company Union Carbide, aqueous dispersions of vinylpyrrolidone, dimethylaminopropylmethacrylamide and lauryldimethylpropyl-methacrylamidoammonium chloride terpolymer, such as Styleze W from ISP, aqueous dispersions of polyurethane/polyacrylic hybrid polymers, such as those sold under the references “Hybridur®” by the company Air Products or “Duromer®” from National Starch, core/shell-type dispersions: for example, those sold by the company Atofina under the reference Kynar (core: fluorinated-shell: acrylic) or else those described in document U.S. Pat. No. 5,188,899 (core; silica-shell: silicone), and mixtures thereof.

b) Nonagueous Dispersion

Mention may also be made of dispersions of particles of a grafted ethylenic, for example acrylic, polymer in a liquid fatty phase, the ethylenic polymer being advantageously dispersed in the absence of any additional stabilizer on the surface of the particles, as described for example in document WO 04/055081.

The composition according to the invention may comprise a plasticizer that promotes the formation of a film with the film-forming polymer. Such a plasticizer may be chosen from all the compounds known to those skilled in the art to be capable of performing the desired function.

In this respect, mention may be made of acrylic dispersions in isododecane, for instance Mexomere PAP® by the company Chimex.

Dyestuff

The composition according to the invention may also comprise at least one dyestuff, for instance pulverulent dyestuffs, liposoluble dyes and water-soluble dyes.

The pulverulent dyestuffs may be chosen from pigments and pearlescent agents.

The pigments may be white or colored, mineral and/or organic, and coated or uncoated. Among the mineral pigments, mention may be made of titanium dioxide, optionally surface-treated, zirconium oxide, zinc oxide or cerium oxide, and also iron oxide or chromium oxide, manganese violet, ultramarine blue, chromium hydrate and ferric blue. Among the organic pigments, mention may be made of carbon black, pigments of D & C type, and lacs based on cochineal carmine or on barium, strontium, calcium or aluminium.

The pearlescent agents may be chosen from white pearlescent pigments such as mica coated with titanium or with bismuth oxychloride, colored pearlescent pigments such as titanium mica with iron oxides, titanium mica with, for example, ferric blue or chromium oxide, titanium mica with an organic pigment of the abovementioned type, and also pearlescent pigments based on bismuth oxychloride.

The liposoluble dyes are, for example, Soudan red, D&C Red 17, D&C Green 6, β-carotene, soybean oil, Soudan brown, D&C Yellow 11, D&C Violet 2, D&C Orange 5, quinoline yellow and annatto.

These dyestuffs may be present at a content ranging from 0.01 to 30% by weight relative to the total weight of the composition.

Fillers

The composition according to the invention may also comprise at least one filler.

The fillers may be chosen from those that are well known to those skilled in the art and commonly used in cosmetic compositions. The fillers may be mineral or organic, and lamellar or spherical. Mention may be made of talc, mica, silica, kaolin, polyamide powders, for instance the Nylon® sold under the name Orgasol® by the company Atochem, poly-β-alanine powders and polyethylene powders, powders of tetrafluoroethylene polymers, for instance Téflon®, lauroyllysine, starch, boron nitride, expanded polymeric hollow microspheres such as those of polyvinylidene chloride/acrylonitrile, for instance the products sold under the name Expancel® by the company Nobel Industrie, acrylic powders such as those sold under the name Polytrap® by the company Dow Corning, poly(methyl methacrylate) particles and silicone resin microbeads (Tospearls® from Toshiba, for example), precipitated calcium carbonate, magnesium carbonate and magnesium hydrocarbonate, hydroxyapatite, hollow silica microspheres (Silica Beads® from Maprecos), glass or ceramic microcapsules, metal soaps derived from organic carboxylic acids having from 8 to 22 carbon atoms, and for example from 12 to 18 carbon atoms, for example zinc stearate, magnesium stearate or lithium stearate, zinc laurate or magnesium myristate, and non-expanded microspheres of vinylidene chloride/acrylonitrile/methyl methacrylate copolymer or of acrylonitrile homopolymer copolymer, for instance those sold, respectively, under the references Expancel® 820 DU 40 and Expancel® 007WU by the company Akzo Nobel.

The fillers may represent from 0.1 to 25%, for example from 1 to 20% by weight, relative to the total weight of the composition.

Fibers

The composition according to the invention may also comprise fibers that for example allow an improvement in the lengthening effect when the composition is a mascara.

The term “fiber” should be understood to mean an object of length L and of diameter D such that L is very much greater than D, D being the diameter of the circle in which the cross section of the fiber is inscribed. In particular, the L/D ratio (or shape factor) is chosen in the range from 3.5 to 2500, for example from 5 to 500, and for example from 5 to 150.

The fibers that can be used in the composition of the invention may be fibers of synthetic or natural, mineral or organic origin. They may be short or long, individual or organized, for example braided, and hollow or solid. They may have any shape, and may for example have a circular or polygonal (square, hexagonal or octagonal) cross section, depending on the intended specific application. Their ends may for example be blunt and/or polished to prevent injury.

The fibers may have for example a length ranging from 1 μm to 10 mm, for example from 0.1 mm to 5 mm, and for example from 0.3 mm to 3.5 mm. Their cross section may be within a circle of diameter ranging from 2 nm to 500 μm, for example ranging from 100 mm to 100 μm, and for example from 1 μm to 50 μm. The weight or yarn count of the fibers is often given in denier or decitex, and represents the weight in grams per 9 km of yarn. The fibers according to the invention may for example have a yarn count chosen in the range from 0.15 to 30 denier, and for example from 0.18 to 18 denier.

The fibers that can be used in the composition of the invention may be chosen from rigid or non-rigid fibers, and may be of synthetic or natural, mineral or organic origin.

Moreover, the fibers may or may not be surface-treated, may be coated or uncoated, and may be colored or uncolored.

As fibers that can be used in the composition according to the invention, mention may be made of non-rigid fibers such as polyamide (Nylon®) fibers or rigid fibers such as polyimideamide fibers, for instance those sold under the names Kermel® and Kermel Tech® by the company Rhodia or poly(p-phenyleneterephthalamide) (or aramide) fibers sold especially under the name Keviar® by the company Dupont de Nemours.

The fibers may be present in the composition according to the invention at a content ranging from 0.01% to 10% by weight, relative to the total weight of the composition, for example from 0.1% to 5% by weight, and for example from 0.3% to 3% by weight.

Cosmetic Active Agents

The composition according to the invention may furthermore comprise all the ingredients conventionally used in cosmetics. These ingredients may particularly be chosen from polymers, especially fixing polymers; hair-conditioning agents; opacifiers, fragrances; thickeners; gelling agents; hair dyes; silicone resins; silicone gums; preserving agents; antioxidants; cosmetic active agents; sunscreens; pH stabilizers; vitamins; moisturizers; antiperspirants; deodorants; self-tanning compounds, and mixtures thereof. The amounts of these various ingredients are those conventionally used in the fields concerned, for example from 0.01% to 20% of the total weight of the composition.

Formulation

The composition according to the invention may be in liquid, pasty, solid, mousse or spray form. It may be an anhydrous composition.

The composition according to the invention may be used for making up human skin, lips and/or keratin fibers. The composition thus finds a particular application as a body or facial makeup composition, such as a foundation, lipstick, lipcare product, nail varnish, nailcare product, mascara or eyeliner; haircare composition, such as a hair dye composition; antisun composition; rinse-off composition to be applied before or after dyeing, bleaching, permanent-waving or relaxing the hair or alternatively between the two steps of a permanent waving or hair relaxing operation; hair composition for holding the hairstyle, such as a styling lacquer, gel, mousse or spray.

According to one exemplary embodiment, the composition may be in the form of lipsticks or of complexion products, for example of the foundation type, or alternatively of a mascara.

Process

In all cases, the compositions according to the invention may be prepared according to methods known to those skilled in the art.

In the case of a composition intended for making up keratin fibers, the process for preparing the compositions according to the invention depends on the type of mascara desired. It also depends in particular on the nature of the wax(es) used.

A subject of the present invention is also a process for making up and/or for the nontherapeutic care of keratin fibers, in which a leave-on composition as defined above is applied to said keratin fibers, for example the eyelashes.

The compositions of the invention may in particular be applied to the eyelashes using a brush or a comb.

The thickening effect of the makeup, using the composition of the invention, may moreover be reinforced by for example selecting the device for applying said composition.

In the present case, it is possible, in the case of making up the eyelashes, to apply said composition with a makeup brush as described in patents FR 2 701 198, FR 2 605 505, EP 792 603 and EP 663 161.

The composition according to the invention may be packaged in a container delimiting at least one compartment that comprises said composition, said container being closed by means of a closing member.

The container may be associated with an applicator, for example in the form of a brush comprising an arrangement of bristles maintained by a twisted wire. Such a twisted brush is for example described in patent U.S. Pat. No. 4,887,622. It may also be in the form of a comb comprising a plurality of application members, obtained for example by moulding. Such combs are described, for example, in patent FR 2 796 529. The applicator may be solidly attached to the container, as described, for example, in patent FR 2 761 959. In one exemplary embodiment, the applicator is solidly attached to a shaft which, itself, is solidly attached to the closing member.

The closing member may be coupled to the container by screwing. Alternatively, the coupling between the closing member and the container takes place other than by screwing, for example via a bayonet mechanism, by click-fastening or by tightening. The term “click-fastening” is for example intended to mean any system involving the passing of a rim or bead of material by elastic deformation of a portion, for example of the closing member, followed by return to the elastically unstressed position of said portion after the rim or bead has been passed.

The container may be at least partly made of a thermoplastic material. By way of examples of thermoplastic materials, mention may be made of polypropylene or polyethylene.

Alternatively, the container may be made of a non-thermoplastic material, for example of glass or metal (or alloy).

The container may be for example equipped with a drainer located in the region of the aperture of the container. Such a drainer makes it possible to wipe the applicator and, optionally, the shaft to which it may be solidly attached. Such a drainer is described, for example, in patent FR 2 792 618.

The content of the patents or patent applications mentioned above are incorporated into the present application by way of reference.

The examples that follow are presented by way of non-limiting illustration of the invention. Unless otherwise indicated, the amounts are given in grams.

The following tests were used to evaluate the waterproof nature.

I—Test for Evaluating the Waterproof Nature and the Makeup Removal by Immersion

Specimen and Makeup Application

False eyelash specimens are made with straight black Caucasian hair with a fringe length of 19 mm. Said fringes are mounted between two 30 mm by 30 mm plates.

The hair is made up with the test composition by effecting three times ten sweeps, separated by an interval of two minutes, using a mascara brush.

The composition is left to dry for one hour at ambient temperature (25° C.).

Description of the Test

The specimen is immersed in water for four minutes. The appearance of degradation of the film (swelling, irregularity, detachment, etc.) is observed visually, and the time corresponding to the start of this phenomenon was noted.

The time t corresponds to the time starting from which the film of mascara on the specimen begins to visually alter.

II—Test for Evaluating the Makeup Removal on Dry Cotton Wool

The specimen used for test I described above is subsequently pinched in a piece of cotton wool for ten seconds, and the cotton wool is then drawn to remove the makeup from said specimen. The efficiency of the makeup removal is then noted by evaluating the amount of mascara on the cotton wool and on the eyelashes.

### EXAMPLE 1

### Wax-Based Mascara

| Composition A | % by weight |
|---|---|
| Carnauba microwax (melting point: 83-86° C.) | 18.30 |
| Hydrogenated C36 diacid/ethylenediamine condensate, | 1.50 |
| esterified with protected stearyl alcohol (Uniclear | |
| 100 VG from Arizona) | |
| Modified distearyldimethylammonium hectorite | 3.60 |
| Black iron oxide | 3.80 |
| Ultramarine blue | 1.90 |
| Water | 7.30 |
| Vinyl acetate/allyl stearate copolymer (65/35) | 6.50 |
| Propylene carbonate | 1.20 |
| White beeswax | 13.00 |
| Isododecane | qs 100 |
| Propyl p-hydroxybenzoate | 0.20 |
| 12-hydroxystearic acid oligomer stearate (Solsperse 21 000 | 0.60 |
| from Uniquema) | |
| Paraffin microwax | 6.50 |

Compositions comprising composition A and also at least one polyelectrolyte and at least one surfactant with an HLB of greater than or equal to 6 at 25° C. are prepared by mixing these components in the proportions given in Table I below.

The results of water resistance and of makeup-removing behavior are given in Table I below.

TABLE I

| | 1 | 2 | 3 | 4 | 5 |
|---|---|---|---|---|---|
| Trials | (comparative) | (comparative) | (invention) | (invention) | (invention) |
| Composition A | 96 | 100 | 88 | 94 | 88 |
| SIMULGEL 600 ® | | | 10 | | 10 |
| COSMEDIA SP ® | 4 | | | 4 | |
| POLYSORBATE 80 | | | 2 | 2 | |
| POLYSORBATE 20 | | | | | 2 |
| Behavior in water | NTR | | t = 30 s | t = 20 s | t = 1 min |
| after immersion in | | | | | 10 s |
| water for 4 min | | | | | |
| (Test I) | | | | | |
| Makeup-removing | VERY | NO | TOTAL | TOTAL | TOTAL |
| behavior after | SLIGHT | MAKEUP | MAKEUP | MAKEUP | MAKEUP |
| pinching/drawing | MAKEUP | REMOVAL | REMOVAL | REMOVAL | REMOVAL |
| once on dry cotton | REMOVAL | | | | |
| wool (Test II) | | | | | |

NTR = Nothing to report
Simulgel 600 ® contains:
4% of acrylamide/sodium acrylamido-2-methylpropanesulphonate copolymer,
0.7% of polysorbate 80,
2.1% of isohexadecane,
2.95% of water,
0.25% of sorbitan oleate.

### EXAMPLE 2

2.1 Microdispersion of Carnauba Wax

A microdispersion of carnauba wax having the following composition is prepared:

| | | |
|---|---|---|
| Carnauba wax | 27.00 | g |
| Polyoxethylenated (30 EO) glyceryl monostearate (Tagat | 6.75 | g |
| S from Goldschmidt) | | |
| Ethanol | 10.00 | g |
| Water | qs 100.00 | g |

The wax and the surfactant are heated to 90° C. while homogenizing the mixture with moderate stirring. The water, heated to 90° C., is then incorporated while continuing the stirring. The mixture is cooled to ambient temperature and the ethanol is added so as to obtain a microdispersion of wax having a mean particle diameter of approximately 170 nm.

| 2.2 Wax-based mascara | % by weight |
|---|---|
| Candelilla wax esterified with polyalkoxyl | 0.92 |
| polydimethylsiloxane (siliconyl candelilla wax from Koster | |
| Keunen) | |
| Carnauba wax | 4.37 |
| Microdispersion of carnauba wax | 7.00 |
| Synthetic beeswax | 3.12 |
| Paraffin wax | 2.10 |
| Highly branched polyolefin wax | 0.10 |
| Polyethylene wax | 1.85 |
| Oxyethylenated (20 EO) sorbitan monooleate (Tween 80 | 1.75 |
| from Uniquema) | |
| Modified distearyldimethylammonium hectorite | 5.80 |
| Hydroxyethylcellulose quaternised with | 0.10 |
| 2,3-epoxypropyltrimethylammonium chloride | |
| Vinyl polylaurate (Mexomere PP from Chimex) | 0.75 |
| Vinyl acetate/allyl stearate copolymer (65/35) | 2.21 |
| Sodium polymethacrylate in water at 25% AM (Darvan | 1.00 |
| No. 7 from Vanderbilt) | |
| Vinyl pyrrolidone/eicosene copolymer | 2.00 |
| Oxyethylenated (20 EO)/oxypropylenated (20 PO) | 0.10 |
| polydimethylsiloxane | |
| Polybutene (MW 2060) | 1.00 |
| Propylene carbonate | 1.94 |
| Rice starch | 1.50 |
| Acrylamide/Na AMPS copolymer in isohexadecane | 1.40 |
| with polysorbate 80 (Simulgel 600 ® from Seppic) | |
| Black iron oxide | 4.19 |
| Water | 2.89 |
| Ethanol | 2.00 |
| Preserving agents | qs |
| Isododecane | qs 100 |

This formula exhibits good water resistance and is easily removed with a makeup remover having an aqueous phase such as the product sold under the reference Effacils.

### EXAMPLE 3

### Influence of the Presence of a Nonionic Surfactant with an HLB of Greater than or Equal to 6 at 25° C.

Composition

TRIALS

| | 11 | 12 | 13 | 14 |
|---|---|---|---|---|
| Composition A | 90 | 90 | 88 | 88 |
| Acrylamide/Na AMPS copolymer in | — | 10 | — | 10 |
| isohexadecane with polysorbate 80, | | | | |
| Simulgel 600 ® from Seppic | | | | |
| Crosslinked starch sodium glycolate, | 10 | — | 10 | — |
| Primojel from Avebe | | | | |
| Polyoxyethylenated (20) sorbitan | — | — | 2 | 2 |
| monooleate, Tween 80 V ® from | | | | |
| Uniquema | | | | |

Results of the Tests for Evaluating the Waterproof Nature and Makeup Removal

| | TRIALS | | | |
|---|---|---|---|---|
| | 11 | 12 | 13 | 14 |
| Time corresponding to | >4 | 3.5 | 2 | 0.5 |
| the appearance of | | | | |
| degradation of the | | | | |
| film (minutes) (test I) | | | | |
| Evaluation of makeup | No trace | Slight | Partial | Total makeup |
| removal on cotton | | makeup | makeup | removal of |
| wool (test II) | | removal in | removal | colorant |
| | | grains | | |

Comparison of trials 11 and 13 and also of trials 12 and 14 shows that the presence of Tween 80 V® very clearly improves the makeup removal.

Although the present invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claimed is:

1. A cosmetic composition for making up keratin materials, the composition comprising:
at least one polyelectrolyte;
at least one surfactant having an HLB of at least about 6 at 25° C.; and
at least one dyestuff;
wherein:
the polyelectrolyte is branched and/or crosslinked;
the composition comprises at least one of water and a water-soluble solvent in an amount of less than about 50% by weight relative to a total weight of the composition; and
the composition comprises an oily continuous phase.

2. The composition according to claim 1, wherein the at least one polyelectrolyte is selected from the group consisting of an acrylamide/2-methyl-2-[(1-oxo-2-propenyl)amino]-1-propanesulphonic acid copolymer, a polyacrylate, and copolymers thereof.

3. The composition according to claim 1, wherein the at least one polyelectrolyte is present in an amount of at least about 0.05% by weight relative to the total weight of the composition.

4. The composition according to claim 1, wherein the at least one polyelectrolyte is present in an amount of from about 0.05 to about 15% by weight relative to the total weight of the composition.

5. The composition according to claim 1, wherein the at least one surfactant is selected from the group consisting of a nonionic surfactant, an ionic surfactant, a surfactant of mixed ionic and nonionic nature, and mixtures thereof.

6. The composition according to claim 1, wherein the at least one surfactant is present in an amount of from about 0.1 to about 30% by weight relative to the total weight of the composition.

7. The composition according to claim 1, wherein the at least one surfactant is selected from the group consisting of oxyethylenated glycerol ethers, oxypropylenated glycerol ethers, oxyethylenated fatty alcohol ethers, oxypropylenated fatty alcohol ethers, fatty acid esters of polyethylene glycol, oxyethylenated fatty acid esters of glycerol ethers, oxypropylenated fatty acid esters of glycerol ethers, oxyethylenated fatty acid esters of sorbitol ethers, oxypropylenated fatty acid esters of sorbitol ethers, dimethicone copolyol, dimethicone copolyol benzoate, copolymers of propylene oxide and ethylene oxide, silicone surfactants, amino acid derivatives, C₁₆-C₃₀ fatty acid salts, polyoxyethylenated fatty acid salts, phosphoric esters, phosphoric ester salts, sulphosuccinates, alkyl ether sulphates, isethionates, acylglutamates, alkylimidazolidiniums, ammonium salts, and mixtures thereof.

8. The composition according to claim 1, wherein the at least one of water and the water-soluble solvent is present in an amount of about 40% by weight or less relative to the total weight of the composition.

9. The composition according to claim 1, further comprising at least one volatile oil.

10. The composition according to claim 9, wherein the at least one volatile oil is present in an amount of from about 20 to about 80% by weight relative to the total weight of the composition.

11. The composition according to claim 9, wherein the at least one volatile oil is selected from the group consisting of hydrocarbon-based oils, silicone oils, fluoro oils, and mixtures thereof.

12. The composition according to claim 1, further comprising a structuring agent selected from the group consisting of waxes and semi-crystalline polymers.

13. The composition according to claim 1, further comprising a film-forming polymer.

14. The composition according to claim 1, further comprising at least one member selected from the group consisting of fillers and fibers.

15. A cosmetic process for making up keratin materials, comprising applying the composition according to claim 1 to the keratin materials.

16. A made-up support, comprising a makeup provided on a support, the makeup comprising the composition according to claim 1.

17. A leave-on cosmetic composition for making up and/or nontherapeutic care of keratin fibers, the composition comprising:
at least one polyelectrolyte; and
at least one surfactant having an HLB of at least about 6 at 25° C.;
wherein:
the polyelectrolyte is branched and/or crosslinked;
the composition comprises at least one of water and a water-soluble solvent in an amount of less than about 50% by weight relative to a total weight of the composition; and
the composition comprises an oily continuous phase.

18. The composition according to claim 17, wherein the at least one polyelectrolyte is selected from the group consisting of an acrylamide/2-methyl-2-[(1-oxo-2-propenyl)amino]-1-propanesulphonic acid copolymer, a polyacrylate, and copolymers thereof.

19. The composition according to claim 17, wherein the at least one polyelectrolyte is present in an amount of at least about 0.05% by weight relative to the total weight of the composition.

20. The composition according to claim 17, wherein the at least one polyelectrolyte is present in an amount of from about 0.05 to about 15% by weight relative to the total weight of the composition.

21. The composition according to claim 17, wherein the at least one surfactant is selected from the group consisting of a nonionic surfactant, an ionic surfactant, a surfactant of mixed ionic and nonionic nature, and mixtures thereof.

22. The composition according to claim 17, wherein the at least one surfactant is present in an amount of from about 0.1 to about 30% by weight relative to the total weight of the composition.

23. The composition according to claim 17, wherein the at least one surfactant is selected from the group consisting of oxyethylenated glycerol ethers, oxypropylenated glycerol ethers, oxyethylenated fatty alcohol ethers, oxypropylenated fatty alcohol ethers, fatty acid esters of polyethylene glycol, oxyethylenated fatty acid esters of glycerol ethers, oxypropylenated fatty acid esters of glycerol ethers, oxyethylenated fatty acid esters of sorbitol ethers, oxypropylenated fatty acid esters of sorbitol ethers, dimethicone copolyol, dimethicone copolyol benzoate, copolymers of propylene oxide and ethylene oxide, silicone surfactants, amino acid derivatives, C₁₆-C₃₀ fatty acid salts, polyoxyethylenated fatty acid salts, phosphoric esters, phosphoric ester salts, sulphosuccinates, alkyl ether sulphates, isethionates, acylglutamates, alkylimidazolidiniums, ammonium salts, and mixtures thereof.

24. The composition according to claim 17, wherein the at least one of water and the water-soluble solvent is present in an amount of about 40% by weight or less relative to the total weight of the composition.

25. The composition according to claim 17, further comprising at least one volatile oil.

26. The composition according to claim 25, wherein the at least one volatile oil is present in an amount of from about 20 to about 80% by weight relative to the total weight of the composition.

27. The composition according to claim 25, wherein the at least one volatile oil is selected from the group consisting of hydrocarbon-based oils, silicone oils, fluoro oils, and mixtures thereof.

28. The composition according to claim 17, further comprising a structuring agent selected from the group consisting of waxes and semi-crystalline polymers.

29. The composition according to claim 17, further comprising a film-forming polymer.

30. The composition according to claim 17, further comprising a dyestuff.

31. The composition according to claim 17, further comprising at least one member selected from the group consisting of fillers and fibers.

32. A cosmetic process for making up and/or nontherapeutic care of keratin fibers, comprising applying the composition according to claim 17 to the keratin fibers.

33. A made-up support, comprising a makeup provided on a support, the makeup comprising the composition according to claim 17.

34. A leave-on cosmetic composition for making up and/or nontherapeutic care of keratin fibers, the composition comprising:
at least one polyelectrolyte; and
at least one surfactant having an HLB of at least about 6 at 25° C.;
wherein:
the polyelectrolyte comprises at least one member selected from the group consisting of an acrylamide/2-methyl-2-[(1-oxo-2-propenyl)amino]-1-propanesulphonic acid copolymer, a polyacrylate, a crosslinked starch glycolate in powdered form, a starch-based grafted copolymer, an ionizable polysaccharide derivative, a polyacrylic acid, a polyacrylic acid alkyl acrylate copolymer, a polyacrylamidomethylpropanesulphonic acid partially neutralized with aqueous anmmonia and highly crosslinked (AMPS), a crosslinked polyoxyethylenated AMPS/alkyl methacrylate copolymer, and a non-crosslinked polyoxyethylenated AMPS/alkyl methacrylate copolymer; and
the composition comprises an oily continuous phase.

35. The composition according to claim 34, wherein the at least one polyelectrolyte is selected from the group consisting of an acrylamide/2-methyl-2-[(1-oxo-2-propenyl)amino]-1-propanesulphonic acid copolymer, a polyacrylate, and copolymers thereof.

36. The composition according to claim 34, wherein the at least one polyelectrolyte is present in an amount of at least about 0.05% by weight relative to a total weight of the composition.

37. The composition according to claim 34, wherein the at least one polyelectrolyte is present in an amount of from about 0.05 to about 15% by weight relative to a total weight of the composition.

38. The composition according to claim 34, wherein the at least one surfactant is selected from the group consisting of a nonionic surfactant, an ionic surfactant, a surfactant of mixed ionic and nonionic nature, and mixtures thereof.

39. The composition according to claim 34, wherein the at least one surfactant is present in an amount of from about 0.1 to about 30% by weight relative to a total weight of the composition.

40. The composition according to claim 34, wherein the at least one surfactant is selected from the group consisting of oxyethylenated glycerol ethers, oxypropylenated glycerol ethers, oxyethylenated fatty alcohol ethers, oxypropylenated fatty alcohol ethers, fatty acid esters of polyethylene glycol, oxyethylenated fatty acid esters of glycerol ethers, oxypropylenated fatty acid esters of glycerol ethers, oxyethylenated fatty acid esters of sorbitol ethers, oxypropylenated fatty acid esters of sorbitol ethers, dimethicone copolyol, dimethicone copolyol benzoate, copolymers of propylene oxide and ethylene oxide, silicone surfactants, amino acid derivatives, C₁₆-C₃₀ fatty acid salts, polyoxyethylenated fatty acid salts, phosphoric esters, phosphoric ester salts, sulphosuccinates, alkyl ether sulphates, isethionates, acylglutamates, alkylimidazolidiniums, ammonium salts, and mixtures thereof.

41. The composition according to claim 34, further comprising at least one of water and a water-soluble solvent in an amount of 40% by weight or less relative to a total weight of the composition.

42. The composition according to claim 34, further comprising at least one volatile oil.

43. The composition according to claim 42, wherein the at least one volatile oil is present in an amount of from about 20 to about 80% by weight relative to a total weight of the composition.

44. The composition according to claim 42, wherein the at least one volatile oil is selected from the group consisting of hydrocarbon-based oils, silicone oils, fluoro oils, and mixtures thereof.

45. The composition according to claim 34, further comprising a structuring agent selected from the group consisting of waxes and semi-crystalline polymers.

46. The composition according to claim 34, further comprising a film-forming polymer.

47. The composition according to claim 34, further comprising a dyestuff.

48. The composition according to claim 34, further comprising at least one member selected from the group consisting of fillers and fibers.

49. A cosmetic process for making up and/or nontherapeutic care of keratin fibers, comprising applying the composition according to claim 34 to the keratin fibers.

50. A made-up support, comprising a makeup provided on a support, the makeup comprising the composition according to claim 34.

51. A method for preparing a cosmetic composition for making up keratin materials, the method comprising combining at least one polyelectrolyte and at least one surfactant having an HLB of at least about 6 at 25° C., wherein:
the composition comprises an oily continuous phase; and
the composition exhibits good water resistance and is easily removed

52. A method for preparing a leave-on cosmetic composition for making up and/or nontherapeutic care of keratin fibers, the method comprising combining at least one polyelectrolyte and at least one surfactant having an HLB of at least about 6 at 25° C., wherein:
the composition comprises an oily continuous phase; and
the composition exhibits good water resistance and is easily removed.
